(19) United States
(12) Patent Application Publication
(10) Pub. No.: US 2019/0216969 A1
(43) Pub. Date: Jul. 18, 2019

(54) METHOD AND SYSTEM FOR INHIBITING MICROBIAL GROWTH BY PHOTOCATALYTIC OXIDATION

(71) Applicant: NEW AIR TECHNOLOGIES, INC., Ivoryton, CT (US)

(72) Inventor: John Carlson, Ivoryton, CT (US)

(73) Assignee: NEW AIR TECHNOLOGIES, INC., Ivoryton, CT (US)

(21) Appl. No.: 16/158,843

(22) Filed: Oct. 12, 2018

Related U.S. Application Data
(60) Provisional application No. 62/571,451, filed on Oct. 12, 2017.

(51) Int. Cl. A61L 9/20; B01J 21/18; B01J 21/06; B01J 27/10; B01J 35/00; A01G 7/06

(52) U.S. Cl. CPC A61L 9/205; B01J 21/18; B01J 21/063; A61L 2209/14; B01J 35/004; A01G 7/06; A61L 2209/12; B01J 27/10

(57) ABSTRACT

A system for inhibiting microbial growth by photocatalytic oxidation includes an intake having a first inlet port and a first outlet port, an air mover having a second inlet port and a second outlet port, and a filter having a third inlet port. An ultraviolet light source is disposed within the filter to promote photocatalytic oxidation of microbiological particles or other microbial growth, bioaerosols, volatile organic compounds or other particulate matter passing through the filter. The filter includes a filter body, a filter channel disposed within the filter body and having an outer surface and an inner surface. A filter media is disposed within the filter channel. A plurality of inlet ports is distributed over the inner surface and a plurality of outlet ports is distributed over the outer surface.

### CROSS-REFERENCE TO RELATED APPLICATION

This application claims the benefit, in accordance with 35 U.S.C. § 119(e), of U.S. Provisional Patent Application Ser. No. 62/571,451; filed on Oct. 12, 2017, which is incorporated herein by reference in its entirety.

### Technical Field

The present invention relates generally to methods and systems for inhibiting microbial growth by photocatalytic oxidation, and more particularly, to methods and systems that utilize photocatalytic oxidation to inhibit microbial growth and to diminish undesirable odor-causing elements emitted by or emanating from the cultivation of cannabis and other plant species.

### BACKGROUND OF THE INVENTION

Various systems are known for inhibiting undesirable odor-causing elements emitted by or emanating from the cultivation of cannabis including the use of ventilation and air filtration systems that exhaust into the atmosphere. Some systems are generally directed to controlling microbiological particles and other organic constituents via the physical removal of such particles by encapsulating and dispersing the particles which can then be flushed away with water or other solvents. In other systems, chemical agents are used to neutralize or provide some other chemical change to the offending particles. Most often, a system will utilize the combination of neutralization with chemical change and physical removal of the particles. Other systems, particularly those directed to controlling or abating offending odorous emissions, typically cover the particles with perfumes or the like or to mask the odor. Still other systems utilize various types of filter media, for example, by promoting the collection of particles in or on material that traps and retains the particles or on surfaces that are ionically charged to attract oppositely charged particles.

### Summary

In one aspect, the present invention is directed to a system for inhibiting microbial growth by photocatalytic oxidation, the system comprising: an intake having a first inlet port and a first outlet port; an air mover having a second inlet port and a second outlet port; a filter having a third inlet port; an ultraviolet light source disposed within the filter to promote photocatalytic oxidation of microbiological particles or other microbial growth, bioaerosols, volatile organic compounds or other particulate matter passing through the filter. In one embodiment, the filter of the present invention comprises: a filter body; a filter channel disposed within the filter body having an outer surface and an inner surface; a filter media disposed within the filter channel; a plurality of inlet ports distributed over the inner surface; and a plurality of outlet ports distributed over the outer surface.

In one aspect, the present invention is directed to a method for inhibiting microbial growth by photocatalytic oxidation, the method comprising: providing an intake having a first inlet port and a first outlet port; providing an air mover having a second inlet port and a second outlet port; providing a filter having a third inlet port; providing an ultraviolet light source disposed within the filter to promote photocatalytic oxidation of microbiological particles or other microbial growth, bioaerosols, volatile organic compounds or other particulate matter passing through the filter.

### BRIEF DESCRIPTION OF THE DRAWINGS

FIG. 1 is a front perspective view of one embodiment of a system for inhibiting microbial growth by photocatalytic oxidation in accordance with the present invention.

FIG. 2A is a front perspective view of the system of FIG. 1 with an intake unit removed therefrom.

FIG. 2B is a front perspective view of the system of FIG. 2A with an air mover unit removed therefrom.

FIG. 3A is a front elevation view of one embodiment of an intake unit of the system of FIG. 1.

FIG. 3B is a side perspective view of the intake unit of FIG. 3A.

FIG. 4A is a front elevation view of one embodiment of an air mover unit of the system of FIG. 1.

FIG. 4B is a top plan view of the air mover unit of FIG. 4A.

FIG. 5A is a top perspective view of one embodiment of a filter unit of the system of FIG. 1.

FIG. 5B is a detail top perspective view of the filter unit of FIG. 5A.

FIG. 5C is a detail top plan view of the filter unit of FIG. 5A.

FIG. 6A is a cross-sectional view of one embodiment of a filter assembly in accordance with the present invention.

FIG. 6B is a detail view of one embodiment of a baffle of the filter assembly of FIG. 6A.

FIG. 6C is a detail view of another embodiment of a baffle of the filter assembly of FIG. 6A.

### DETAILED DESCRIPTION OF THE PREFERRED EMBODIMENTS OF INVENTION

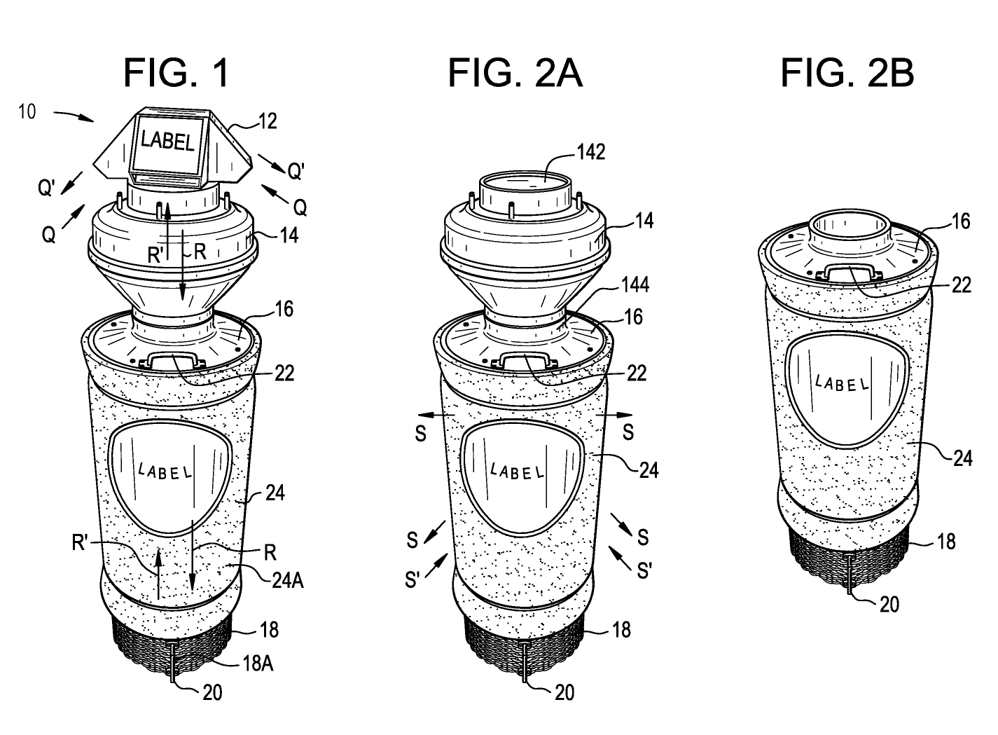

As is shown in FIG. 1, a system for inhibiting microbial growth by photocatalytic oxidation to diminish undesirable odor-causing elements emitted by or emanating from the cultivation of cannabis is designated generally by the reference numeral 10 and is hereinafter referred to as “system 10.” The system 10 includes an intake or intake unit 12, an air mover or air mover unit 14 and a filter or filter unit 16. While the intake unit 12, air mover unit 14 and filter unit 16 are shown and described as separate units, the present invention is not limited in this regard as one unit may provide more than one of the functions provided by the intake, air mover and filter, without departing from the broader aspects of the present invention.

In one embodiment, the intake unit 12 is removeably mounted to the air mover unit 14; and the air mover unit 14 is removeably mounted to the filter unit 16. In one embodiment, the system 10 includes a base 18 and the filter unit 16 extends from the air mover unit 14 to the base 18. In one embodiment, the system 10 includes one or more base handles 20. In one embodiment, the system 10 includes one or more filter unit handles 22. In one embodiment, the system 10 includes a fibrous wrap 24 such that the filter unit 16 is wrapped with a fibrous material 24A. In one embodiment, the system 10 defines a generally upward or upwardly airflow path. Air is drawn into the system 10 by the air mover unit 14 through the intake unit 12, generally upwardly from a floor level or ground level in a direction indicated by the arrows Q, into and through the air mover unit 14 and into and within the filter unit 16 generally downwardly in a direction indicated by the arrow R. Air is exhausted from the system 10 by the air mover unit 14 through the filter unit 16, and the fibrous wrap 24 if present, radially outwardly in a direction indicated by the arrows S.

In one embodiment, the system 10 defines a generally downward or downwardly airflow path. Air is drawn into the system 10 by the air mover unit 16, by reversing the orientation or flowpath of the air mover unit 16, through the filter unit 16, and the fibrous wrap 24 if present, radially inwardly in a direction indicated by the arrows S′. The air flows into and through the air mover unit 14 and into and through the intake unit 12, generally upwardly in a direction indicated by the arrows R′, and is exhausted from the system 10 by the air mover unit 14 through the intake unit 12 in a direction indicated by the arrows Q′. In one embodiment, the system 10 is positioned substantially on a floor or ground level. In one embodiment, the system 10 is positioned or mounted such that the system 10 is elevated off of the floor level or ground by one to twelve inches or by one to twelve feet.

While the filter unit 16 is shown and described as having a cylindrical configuration, the present invention is not limited in this regard as the filter unit 16 may define any geometric configuration, such as for example, square, rectangular, or other polygon configuration, without departing from the broader aspects of the present invention.

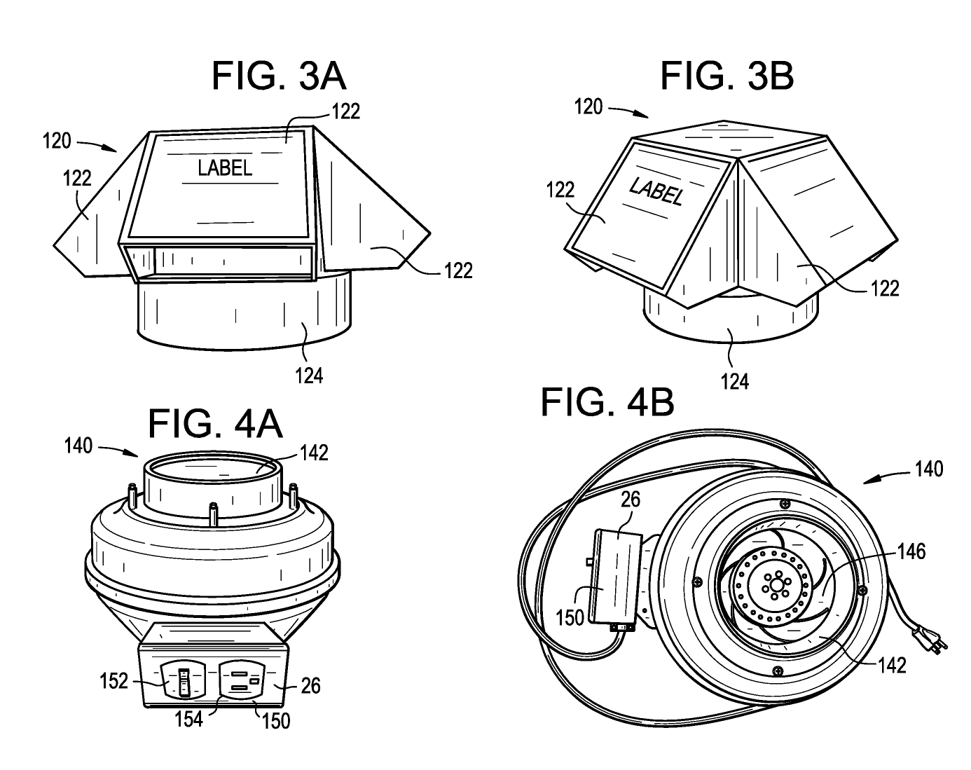

The system 10 is shown in FIG. 2A with the intake unit 12 removed; and in FIG. 2B with both the intake unit 12 and the air mover unit 14 removed. In one embodiment and as shown in FIGS. 3A and 3B, the intake 12 is an intake assembly 120 having one or more inlet ports 122 and an outlet port 124. In one embodiment and as shown in FIGS. 4A and 4B, the air mover unit 14 is air mover assembly 140 having an inlet port 142, an outlet port 144 (FIG. 2A), and a set of blades 146. In one embodiment, a screen or mesh 148 is positioned within the air mover inlet port 144. In one embodiment, the intake outlet port 124 is configured to receive and engage the air mover inlet port 142, for example, by a slip fit. In one embodiment, the air mover assembly 140 is sized to provide an airflow in the range of about 100 cubic feet per minute (“CFM”) to about 1000 CFM. In one embodiment, the air mover assembly 140 is sized to provide an airflow in the range of about 200 CFM to about 400 CFM. In one embodiment, the air mover assembly 140 is sized to provide an airflow in the range of about 250 CFM to about 300 CFM.

As shown in FIGS. 4A and 4B, the system 10 includes a power supply 26. In one embodiment, the power supply 26 is provided with the air mover assembly 140 as an electrical power junction box 150. In one embodiment, the power junction box 150 provides power to the air mover assembly 140 via an electrical switch 152. In one embodiment, the power junction box 150 includes a power connector receptacle 154 from which power can be drawn. In one embodiment, the power supply 26 is configured to receive and distribute standard electric power at a nominal voltage of 120 volts. In one embodiment, the power supply 26 is configured to receive and distribute power generated and/or stored by alternative power production and storage methods including turbine generators, batteries, fuel cells, solar cells, wind turbines, and other like devices or methods.

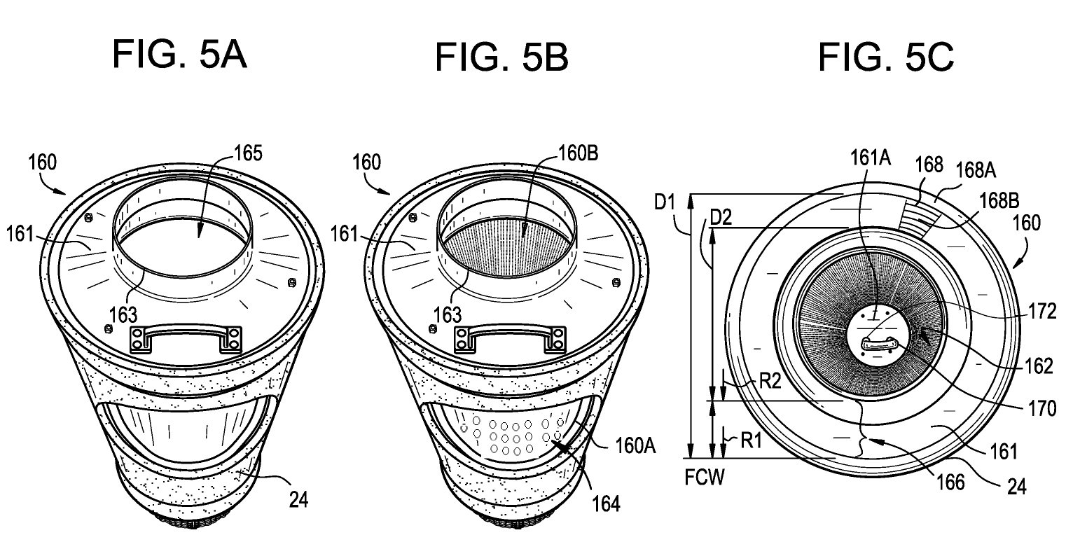

In one embodiment and as shown in FIGS. 2B, 5A, 5B and 5C, the filter unit 16 is a filter unit assembly 160 that includes a filter body 161 having an inlet port 163, a plurality of filter channel inlet ports 162, and a plurality of filter channel outlet ports 164. In one embodiment, the plurality of filter channel outlet ports 164 are covered by the fibrous wrap 24. In one embodiment, the air mover outlet port 144 is configured to receive and engage the filter body inlet port 163, for example, by a slip fit.

As further shown in FIGS. 5A to 5C, the filter body 161 includes a filter channel 166 having an outer surface 160A and an inner surface 160B. The outer surface 160A has a first diameter D1 that defines a corresponding first radius R1. The inner surface 160B has a second diameter D2 that defines a corresponding second radius R2. The difference between the first radius R1 and the second radius R2 defines a filter channel width FCW into which a filter media 168 is disposed. The second diameter D2 defines a filter chamber 165 within the filter body 161. In one embodiment, the plurality of filter channel inlet ports 162 are distributed substantially evenly over substantially the entire inner surface 160B to promote substantially even distribution of microbiological particles and other particulate matter to the filter media 168 disposed in the filter channel 166. In one embodiment, the plurality of filter channel outlet ports 164 are distributed substantially evenly over substantially the entire outer surface 160A to promote substantially even distribution of the filtered air into the surrounding environment. In an embodiment that includes the fibrous wrap 24, the fibrous wrap 24 is configured to capture any particulate matter that otherwise would be ejected through an outlet port 164.

The filter media 168 provides a material substrate onto which microbiological particles or other microbial growth can be adsorbed. In one embodiment, the filter media 168 is an activated charcoal filter 168A wherein microbiological particles or other microbial growth are adsorbed onto the surface of the filter 168A. In one embodiment, the filter media 168 is a salt compound, such as for example a Himalayan salt. In one embodiment, the filter media 168 is treated and configured to trap and eliminate odors and particulate matter in the air passing through the system 10. Thus, in one embodiment, the filter channel width FCW is configured to receive a two-inch nominal filter media 168 therein.

In one embodiment, the filter media 168 is treated, coated or infused, or the like, (hereinafter collectively referred to as “treated”) with an active oxidizing agent. In one embodiment, the oxidizing agent is anatase titanium dioxide (“anatase TiO₂”). Thus, in one embodiment, the filter media 168 is a TiO₂-treated filter 168B which includes the activated charcoal filter 168A treated with the active oxidizing anatase TiO₂. In one embodiment, the filter media 168 is treated with a two-coat system that includes a base or bonding coating and an active oxidizing coating. In one embodiment, the base or bonding coating is rutile titanium dioxide (“rutile TiO₂”). In one embodiment, the active oxidizing is anatase TiO₂. Thus, in one embodiment, the filter media 168 is a TiO₂-treated filter 168B which includes the activated charcoal filter 168A treated with a two-coat system that includes the base or bonding coating of rutile TiO₂ and the active oxidizing coating of anatase TiO₂.

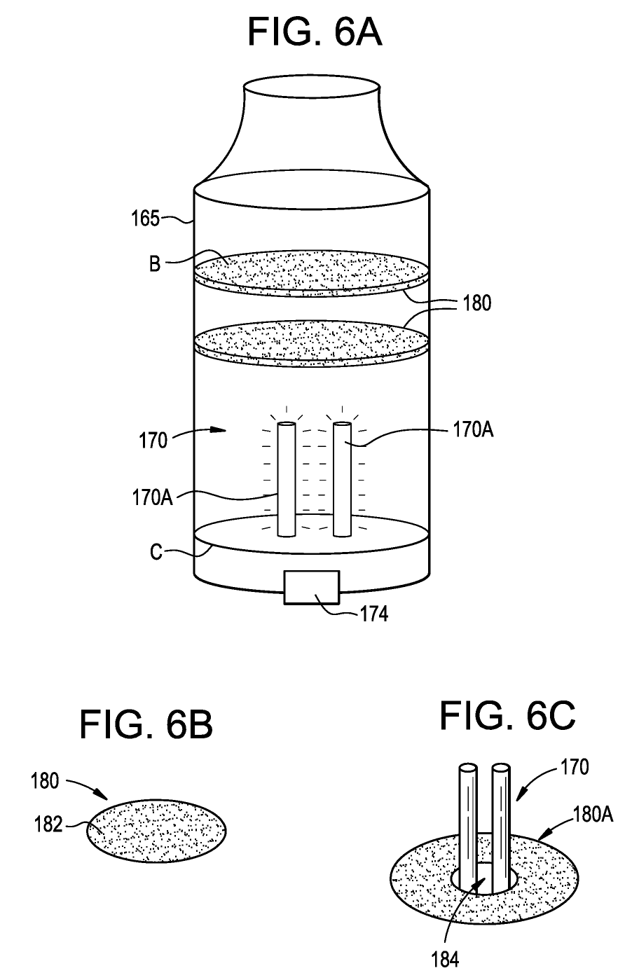

As shown in FIGS. 5C and 6A to 6C, the filter unit assembly 160 further includes an ultraviolet (“UV”) light source 170 to promote photocatalytic oxidation of microbiological particles or other microbial growth, bioaerosols, volatile organic compounds (“VOCs”) and other particulate matter. The UV light source 170 in the presence of an active oxidizing agent creates a photocatalytic oxidation environment within the filter unit assembly 160. During the process of photocatalytic oxidation, hydrogen peroxide, hydroxyl radicals, and hydroxides attach themselves to such organisms and particulates and kill them. In one embodiment, the UV light source 170 is powered by the power supply 26. In one embodiment, the UV light source 170 is a UV light bulb 170A. In one embodiment, the UV light source 170 includes more than one UV light bulb 170A. In one embodiment, the UV light source 170 emits UV light in the UVA wavelength range (i.e., between 315 and 400 nanometers (“nm”). In one embodiment, a UVA lamp 172 is positioned on a bottom surface 161A of the filter body 161. The UV light source 170 excites the anatase TiO₂ to facilitate or promote the oxidation of material.

When exposed to the UV light source 170, the electrons of the anatase TiO₂ are excited from their ambient energy levels to increased energy levels, which thereby allows for the generation of super oxide ions and hydroxyl radicals. The interactions of super oxide ions and hydroxyl radicals with organic matter facilitate the oxidation of the organic matter. Thus, the microbiological particles or other microbial growth adsorbed onto the surface of a TiO₂-coated filter media 168, such as for example the TiO₂-coated filter 168B, and exposed to the UV light source 170, such as for example the UVA lamp 172, the organic matter is effectively and efficiently oxidized and broken down into less offensive matter. Such less offensive matter may be, but is not limited to, carbon dioxide, water, and the like.

In one embodiment, the UV light source 170 includes a ballast unit 174 positioned below the filter body 161 that extends through the bottom surface 161A of the filter body 161 to provide a socket connection for the UV light bulb 170A. In such an embodiment, the base 18 is configured to provide a ballast unit guard 18A (FIGS. 1, 2A and 2B).

In one embodiment and as further shown in FIGS. 6A to 6C, one or more baffles 180 are positioned within the filter chamber 165. The baffles 180 impede or slow down the movement of air along the airflow path to provide a longer dwell time of the air within the photocatalytic oxidation environment. The baffles 180 include a number of perforations 182 through which air passes through the baffle along the airflow path. In one embodiment, the baffle 180 defines an outer diameter substantially equal to the second diameter D2 of the inner surface 160B of filter body 161. In one embodiment, a baffle 180A includes a cutout or hole 184 to accommodate the UV light source 170 such that the baffle 180A can be positioned proximate to a bottom portion of the filter body 161 and over the UV light source 170.

Although this invention has been shown and described with respect to the detailed embodiments thereof, it will be understood by those of skill in the art that various changes may be made and equivalents may be substituted for elements thereof without departing from the scope of the invention. In addition, modifications may be made to adapt a particular situation or material to the teachings of the invention without departing from the essential scope thereof. Therefore, it is intended that the invention not be limited to the particular embodiments disclosed in the above detailed description, but that the invention will include all embodiments falling within the scope of the appended claims.

What is claimed is:

1. A system for inhibiting microbial growth by photocatalytic oxidation, the system comprising:
an intake having a first inlet port and a first outlet port;
an air mover having a second inlet port and a second outlet port;
a filter having a third inlet port; and
an ultraviolet light source disposed within the filter to promote photocatalytic oxidation of microbiological particles or other microbial growth, bioaerosols, volatile organic compounds or other particulate matter passing through the filter.

2. The system of claim 1, further comprising:
a fibrous wrap disposed around the filter.

3. The system of claim 1, the filter comprising:
a filter body;
a filter channel disposed within the filter body having an outer surface and an inner surface;
a filter media disposed within the filter channel;
a plurality of inlet ports distributed over the inner surface; and
a plurality of outlet ports distributed over the outer surface.

4. The system of claim 3, the filter media comprising:
an activated charcoal filter.

5. The system of claim 3, the filter media comprising:
Himalayan salt.

6. The system of claim 3, the filter media comprising:
an activated charcoal filter treated with an active oxidizing agent.

7. The system of claim 3, the filter media comprising:
an activated charcoal filter treated with anatase titanium dioxide.

8. The system of claim 3, the filter media comprising:
an activated charcoal filter treated with a two-coat system that includes a base coating of rutile titanium dioxide and an active oxidizing coating of anatase titanium dioxide.

9. The system of claim 1, further comprising:
at least one baffle disposed within the filter.

10. A method for inhibiting microbial growth by photocatalytic oxidation, the method comprising:
providing an intake having a first inlet port and a first outlet port;
providing an air mover having a second inlet port and a second outlet port;
providing a filter having a third inlet port;
providing an ultraviolet light source disposed within the filter to promote photocatalytic oxidation of microbiological particles or other microbial growth, bioaerosols, volatile organic compounds or other particulate matter passing through the filter.

11. The method of claim 10, further comprising:
wrapping a fibrous wrap disposed around the filter.

12. The method of claim 10, wherein providing the filter unit comprises:
providing a filter body having a filter channel disposed within the filter body having an outer surface and an inner surface;
positioning a filter media within the filter channel between a plurality of inlet ports distributed over the inner surface and a plurality of outlet ports distributed over the outer surface.

13. The method of claim 12, the filter media comprising:
an activated charcoal filter.

14. The system of claim 12, the filter media comprising:
Himalayan salt.

15. The method of claim 12, the filter media comprising:
an activated charcoal filter treated with an active oxidizing agent.

16. The method of claim 12, the filter media comprising:
an activated charcoal filter treated with a two-coat system that includes a base coating of rutile titanium dioxide and an active oxidizing coating of anatase titanium dioxide.

17. The method of claim 10, further comprising:
positioning at least one baffle within the filter.
